(19) United States
(12) Patent Application Publication
(10) Pub. No.: US 2024/0254189 A1
(43) Pub. Date: Aug. 1, 2024

(54) ULTRAHIGH-AFFINITY SMALL PROTEIN TARGETING PD-L1 AND USE

(71) Applicant: CHINESE PLA GENERAL HOSPITAL, Beijing (CN)

(72) Inventors: Lei ZHAO, Beijing (CN); Yi HU, Beijing (CN); Fan ZHANG, Beijing (CN)

(21) Appl. No.: 18/575,793

(22) PCT Filed: Aug. 12, 2022
(86) PCT No.: PCT/CN2022/112248

§ 371 Date: Feb. 13, 2024

(30) Foreign Application Priority Data
Aug. 13, 2021 (CN) 202110932884.4

(51) Int. Cl. C07K 14/705; A61K 38/00; A61K 47/64; C12N 15/62

(52) U.S. Cl. CPC C07K 14/70503; A61K 47/64; C12N 15/62; A61K 38/00; C07K 2319/30

(57) ABSTRACT

Provided are a class of ultrahigh-affinity small proteins targeting PD-L1 and the use. A class of binding proteins can target PD-L1 and has an ultrahigh affinity. The proteins can competitively bind to wild-type PD-1. The affinity to PD-L1 is much higher than the affinity of the wild-type PD-1 to PD-L1. A fusion protein contains the ultrahigh-affinity protein targeting PD-L1 is also provided.

### TECHNICAL FIELD

The present invention belongs to the field of biotechnology and medicine, and specifically relates to an ultrahigh-affinity small protein targeting PD-L1 and a fusion protein thereof.

### BACKGROUND

The PD-1/PD-L1 signaling pathway is one of the important signaling pathways for the immunity regulation and immunosuppressive effects in the body. Blockade of the PD-1/PD-L1 immunosuppressive signal has become one of the important strategies for current anti-tumor therapies.

However, due to that the blockade of PD-1/PD-L1 immunosuppressive signal currently utilizes monoclonal antibody technology, it cannot achieve a complete coverage of the PD-1/PD-L1 interaction surface. More importantly, although PD-L1 antibodies such as avelumab, durvalumab, and atezolizumab can block the binding of PD-1/PD-L1, their efficacy in clinical trials and treatments varies due to the different binding sites they block.

The binding epitope of an antibody is one of the important factors affecting its therapeutic efficacy. Although avelumab has similar binding sites and higher affinity compared to durvalumab and atezolizumab, the phase III clinical trials thereof in lung and gastric cancers all failed.

These data indicate that subtle differences in PD-L1 antibody binding epitopes are likely to have a significant impact on their therapeutic efficacy. Therefore, how to block the binding of PD-1/PD-L1 more effectively and thereby suppress the PD-1/PD-L1 immunosuppressive signal more effectively is currently an urgent problem to be solved.

In addition, the expression level of PD-L1 is one of the important prognostic indicators for PD-1/PD-L1 antibody therapy.

In summary, there is an urgent need in this field to develop a drug that can block the binding of PD-1/PD-L1 more efficiently and thereby suppressing the PD-1/PD-L1 immunosuppressive signal more effectively, as well as a candidate drug for more accurate and dynamic detection of tumor PD-L1 expression.

### SUMMARY OF THE INVENTION

The purpose of the present invention is to provide a ultrahigh-affinity small protein targeting PD-L1 that is able to blocking the binding of PD-1/PD-L1 more efficiently.

Another purpose of the present invention is to provide a fusion protein based on the ultrahigh-affinity small protein targeting PD-L1 and the preparation method thereof.

In the first aspect of the present invention, it provides a small protein targeting PD-L1, wherein the small protein specifically targets PD-L1 with an ultrahigh affinity, and competes with wild-type PD-1 for binding to PD-L1 and effectively blocks binding of PD-1 to PD-L1.

In another preferred embodiment, the small protein consists of a peptide chain which mainly forms three secondary structures of α-helix.

In another preferred embodiment, the amino acid sequence of the small protein is shown in SEQ ID NOs: 1, 3, 5 or 7.

The present invention further provides a recombinant protein comprising two or more small proteins targeting PD-L1 according to the present invention in tandem.

In the second aspect of the present invention, it provides a fusion protein which comprises a first polypeptide and/or a second polypeptide;
wherein the first polypeptide has a structure as shown in Formula I from N-terminus to C-terminus, and the second polypeptide has a structure as shown in Formula II from N-terminus to C-terminus,

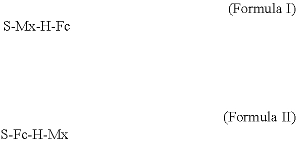

wherein,
S is absent or a signal peptide sequence;
M is a PD-L1 binding domain (or binding element), the amino acid sequence of which is derived from the amino acid sequence of the small protein targeting PD-L1 of the first aspect;
H is a hinge region;
Fc is absent or a constant region of an immunoglobulin, or a fragment thereof;
“-” is a peptide bond or a linking peptide connecting the above elements;
X is a positive integer selected from 1-4.

In another preferred embodiment, “derived from the amino acid sequence of the small protein targeting PD-L1” means that the amino acid sequence of the PD-L1 binding domain (or binding element) is identical or substantially identical to the amino acid sequence of the small protein targeting PD-L1 (i.e., having a homology ≥90%, preferably ≥95%, more preferably ≥98%), and the PD-L1 binding domain (or binding element) retains an activity to bind wild-type PD-L1 (preferably, retains ≥70% binding activity, more preferably ≥80%).

In another preferred embodiment, the amino acid sequence of S is selected from the group consisting of:
(i) a sequence as shown in SEQ ID NO: 21; and
(ii) an amino acid sequence obtained by replacing, deleting, altering or inserting one or more amino acid residues, or adding 1-10 amino acid residues, preferably 1-5 amino acid residues on the basis of SEQ ID NO: 1.

In another preferred embodiment, a nucleotide sequence encoding S is shown in SEQ ID NO: 22.

In another preferred embodiment, the fusion protein is a monomer or a dimer.

In another preferred embodiment, the fusion protein is a homodimer or a heterodimer.

In another preferred embodiment, a disulfide bond can be formed between a first polypeptide and another first polypeptide, between a second polypeptide and another second polypeptide, or between a first polypeptide and a second polypeptide, through cysteines (C) on their Fc region respectively.

In another preferred embodiment, the dimer is selected from the group consisting of: a homodimer formed by two first polypeptides, a homodimer formed by two second polypeptides, or a heterodimer formed by the first polypeptide and the second polypeptide.

In another preferred embodiment, the fusion protein is a homodimer formed by two first polypeptides.

In another preferred embodiment, the sequence of M is set forth in SEQ ID NOs: 1, 3, 5, or 7.

In another preferred embodiment, x is 1, 2, 3, or 4, preferably 2.

In another preferred embodiment, H is a hinge region of human immunoglobulin.

In another preferred embodiment, the human immunoglobulin is selected from the group consisting of: IgG1, IgG4, or a combination thereof.

In another preferred embodiment, the human immunoglobulin is IgG1.

In another preferred embodiment, the amino acid sequence of H is selected from the group consisting of:
(i) a sequence as shown in SEQ ID NO: 9; and
(ii) an amino acid sequence obtained by replacing, deleting, altering or inserting one or more amino acid residues, or adding 1-10 amino acid residues, preferably 1-5 amino acid residues on the basis of SEQ ID NO: 9.

In another preferred embodiment, a nucleotide sequence encoding H is shown in SEQ ID NO: 10.

In another preferred embodiment, Fc is a constant region of a human immunoglobulin, or a fragment thereof.

In another preferred embodiment, Fc is a tandem sequence of CH2 and CH3 regions of a human immunoglobulin, or a single CH3 region of a human immunoglobulin.

In another preferred embodiment, the amino acid sequence of Fc is selected from the group consisting of:
(i) a sequence as shown in SEQ ID NO: 11; and
(ii) an amino acid sequence obtained by replacing, deleting, altering or inserting one or more amino acid residues, or adding 1-30 amino acid residues, preferably 1-10 amino acid residues, more preferably 1-5 amino acid residues on the basis of SEQ ID NO: 11.

In another preferred embodiment, a nucleotide sequence encoding Fc is shown in SEQ ID NO: 12.

In another preferred embodiment, the amino acid sequence of the first polypeptide is selected from the group consisting of:
(i) a sequence as shown in SEQ ID NOs: 13, 15, 17 or 19; and
(ii) an amino acid sequence obtained by replacing, deleting, altering or inserting one or more amino acid residues, or adding 1-30 amino acid residues, preferably 1-10 amino acid residues, more preferably 1-5 amino acid residues on the basis of SEQ ID NOs: 13, 15, 17 or 19.

In another preferred embodiment, a nucleotide sequence encoding the first polypeptide is shown in SEQ ID NOs: 14, 16, 18 or 20.

In another preferred embodiment, the amino acid sequence of the first polypeptide is shown in SEQ ID NO: 13, and the nucleotide sequence encoding the first polypeptide is shown in SEQ ID NO: 14.

In the third aspect of the present invention, it provides a polynucleotide encoding the small protein targeting PD-L1 or recombinant protein according to the first aspect of the present invention or the fusion protein according to the second aspect of the present invention.

In another preferred embodiment, the sequence of the polynucleotide is shown in SEQ ID NOs: 2, 4, 6, 8, 14, 16, 18 or 20.

In another preferred embodiment, the sequence of the polynucleotide is shown in SEQ ID NOs: 4 or 14.

In the fourth aspect of the present invention, it provides a vector comprising the polynucleotide according to the third aspect of the present invention.

In another preferred embodiment, the vector is a PET vector, a pGEM-T vector, a pcDNA3.1, or a combination thereof.

In the fifth aspect of the present invention, it provides a host cell, wherein the host cell comprises the vector according to the fourth aspect of the present invention, or has the polynucleotide according to the third aspect of the present invention integrated into its genome.

In the sixth aspect of the present invention, it provides an immunoconjugate which comprises:
(a) the small protein targeting PD-L1 according to the first aspect of the present invention or a tandem recombinant protein thereof or the fusion protein according to the second aspect of the present invention; and
(b) a coupling moiety selected from a group consisting of a detectable marker, a drug, a toxin, a cytokine, a radionuclide, or an enzyme.

In another preferred embodiment, the coupling moiety is a drug or a toxin.

In another preferred embodiment, the coupling moiety is a detectable marker.

In another preferred embodiment, the coupling moiety is selected from the group consisting of: a fluorescent or luminescent marker, radioactive marker, MRI (magnetic resonance imaging) or CT (computed tomography) contrast agent.

In the seventh aspect of the present invention, it provides a pharmaceutical composition which comprises:
(a) the small protein targeting PD-L1 or the recombinant protein thereof according to the first aspect of the present invention or the fusion protein according to the second aspect of the present invention, or an encoding gene thereof; or the immunoconjugate according to the sixth aspect; and
(b) a pharmaceutically acceptable carrier.

In another preferred embodiment, the pharmaceutical composition is used for tracing or treating a tumor expressing PD-L1 protein (i.e. PD-L1 positive).

In another preferred embodiment, the content of component (a) is 0.1-99.9 wt %, preferably 10-99.9 wt %, more preferably 70%-99.9 wt %.

In another preferred embodiment, the pharmaceutical composition is an oral dosage form, an injection form, or an topical drug dosage form.

In another preferred embodiment, the dosage form of the pharmaceutical composition includes tablet, granule, capsule, oral liquid, or injection.

In another preferred embodiment, the pharmaceutical composition or formulation is selected from the group consisting of: a suspension formulation, a liquid formulation, or a lyophilized formulation.

In another preferred embodiment, the liquid formulation is an aqueous injection formulation.

In another preferred embodiment, the liquid formulation has a shelf life of one to three years, preferably one to two years, more preferably one year.

In another preferred embodiment, the liquid formulation has a storage temperature of from 0° C. to 16° C., preferably from 0° C. to 10° C., more preferably from 2° C. to 8° C.

In another preferred embodiment, the lyophilized formulation has a shelf life of from six months to two years, preferably from six months to one year, more preferably half a year.

In another preferred embodiment, the lyophilized formulation has a storage temperature of ≤42° C., preferably ≤37° C., more preferably ≤30° C.

In another preferred embodiment, the pharmaceutically acceptable carrier comprises: a surfactant, a solution stabilizer, an isotonicity adjusting agent, a buffer, or a combination thereof.

In another preferred embodiment, the pharmaceutically acceptable carrier is selected from the group consisting of: an infusion carrier and/or injection carrier, preferably one or more carriers selected from the following group: a normal saline, glucose saline, or a combination thereof.

In another preferred embodiment, the solution stabilizer is selected from a group consisting of a saccharide solution stabilizer, an amino acid solution stabilizer, an alcohol solution stabilizer, or a combination thereof.

In another preferred embodiment, the saccharide solution stabilizer is selected from a group consisting of a reducing saccharide solution stabilizer or a non-reducing saccharide solution stabilizer.

In another preferred embodiment, the amino acid solution stabilizer is selected from a group consisting of monosodium glutamate or histidine.

In another preferred embodiment, the alcohol solution stabilizer is selected from a group consisting of tri-alcohols, higher saccharide alcohols, propylene glycol, polyethylene glycols, or combinations thereof.

In another preferred embodiment, the isotonicity adjusting agent is selected from a group consisting of sodium chloride or mannitol.

In another preferred embodiment, the buffer is selected from a group consisting of TRIS, histidine buffer, phosphate buffer, or a combination thereof.

In another preferred embodiment, the pharmaceutical composition or formulation is administered to a human or non-human animal.

In another preferred embodiment, the non-human animal comprises: a rodent (such as a rat, a mouse), a primate (such as a monkey).

In another preferred embodiment, the pharmaceutical composition or formulation is administered in an amount of from 0.01 g to 10 g per day, preferably from 0.05 g to 5000 mg per day, more preferably from 0.1 g to 3000 mg per day.

In another preferred embodiment, the pharmaceutical composition or formulation is used for inhibiting and/or treating a tumor.

In another preferred embodiment, the inhibiting and/or treating a tumor comprises a delay associated with the development of symptoms associated with tumor growth and/or a decrease in the severity of such symptoms.

In another preferred embodiment, the inhibiting and/or treating a tumor further comprises a reduction of the pre-existing symptoms accompanying tumor growth and prevention of the appearance of other symptoms.

In another preferred embodiment, the pharmaceutical composition or formulation can be administered in combination with other antineoplastic agents for the treatment of tumors.

In another preferred embodiment, the antineoplastic agent co-administered is selected from the group consisting of: a cytotoxic drug, a hormone antiestrogen, a biological response modifier, a monoclonal antibody, or some other drugs the mechanism of which is currently unknown and needs to be further studied.

In another preferred embodiment, the cytotoxic drug comprises: a drug that acts on the chemical structure of DNA, a drug that affects nucleic acid synthesis, a drug that acts on nucleic acid transcription, a drug that acts mainly on tubulin synthesis, or other cytotoxic drugs.

In another preferred embodiment, the drug that acts on the chemical structure of DNA comprises: an alkylating agent such as nitrogen mustard, nitrosour, a methylsulfonate; a platinum compound such as cisplatin, carboplatin, platinum oxalate; Mitomycin (MMC).

In another preferred embodiment, the drug that affects nucleic acid synthesis comprises: dihydrofolate reductase inhibitors such as methotrexate (MTX) and Alimta, etc.; thymidine synthase inhibitors such as fluorouracil (5FU, FT-207, capecitabine) etc.; purine nucleoside synthase inhibitors such as 6-mercaptopurine (6-MP) and 6-TG etc.; nucleoside reductase inhibitors such as hydroxyurea (HU) etc.; DNA polymerase inhibition agents such as cytarabine (Ara-C) and Gemz, etc.

In another preferred embodiment, the drug that acts on nucleic acid transcription comprises: a drug that selectively acts on a DNA template and inhibits DNA-dependent RNA polymerase, thereby inhibiting RNA synthesis, such as actinomycin D, daunorubicin, Doxorubicin, epirubicin, aclarithromycin, Clomithromycin, and the like.

In another preferred embodiment, the drug that acts mainly on tubulin synthesis comprises: paclitaxel, taxotere, vinblastine, vinorelbine, podophyllum, homoharringtonine.

In another preferred embodiment, the other cytotoxic drug comprises asparaginase that mainly inhibits synthesis of proteins.

In another preferred embodiment, the hormonal antiestrogens comprise: tamoxifen, droloxifene, exemestane, etc.; aromatase inhibitors: aminoglutethimide, lentaron, letrozole, Anastrozole etc.; antiandrogen: Fluoramide RH-LH agonist/antagonist: Zoladex, enatone and so on.

In another preferred embodiment, the biological response modifier comprises: interferon; interleukin-2; thymosin.

In another preferred embodiment, the monoclonal antibodies comprises: MabThera; Cetuximab (C225); Herceptin (Trastuzumab); Bevacizumab (Avastin); Yervoy (Ipilimumab); Nivolumab (OPDIVO); Pembrolizumab (Keytruda); Atezolizumab (Tecentriq).

In the eight aspect of the present invention, it provides a method for preparing the small protein targeting PD-L1 or the recombinant protein thereof or the fusion protein thereof of the present invention, which comprises the following steps:
(i) culturing the host cell according to the fifth aspect of the present invention under a suitable condition, thereby obtaining a culture comprising the small protein or the recombinant protein thereof or the fusion protein; and
(ii) purifying and/or separating the culture obtained in step (i), thereby obtaining the small protein targeting PD-L1 or the recombinant protein thereof or the fusion protein.

In the ninth aspect of the present invention, it provides a use of the small protein targeting PD-L1 or the recombinant protein thereof according to the first aspect of the present invention, or the fusion protein according to the second aspect, or the immunoconjugate according to the sixth aspect, in the preparation of a drug, a reagent, a detection plate, or a kit; wherein the reagent, detection plate or kit is used for detecting PD-L1 in a sample; and the drug is used for treating or preventing a tumor expressing PD-L1 (i.e. PD-L1 positive).

In another preferred embodiment, the reagent is one or more reagents selected from the group consisting of: isotope tracer, contrast agent, flow cytometry reagent, cellular immunofluorescence detection reagent, nano magnetic particle, and imaging agent.

In another preferred embodiment, the reagent used for detecting PD-L1 in the sample is a contrast agent for detecting PD-L1 molecule (in vivo).

In another preferred embodiment, the detection is in vivo detection or in vitro detection.

In another preferred embodiment, the detection includes flow cytometry, cellular immunofluorescence detection, or a combination thereof.

In another preferred embodiment, the agent is used to block the interaction between PD-1 and PD-L1.

In another preferred embodiment, the tumor is a tumor expressing PD-L1 protein (i.e. PD-L1 positive).

In another preferred embodiment, the tumor includes, but is not limited to: acute myelocytic leukemia leukemia, chronic myelocytic leukemia, multiple myelopathy, non-Hodgkin lymphoma, colorectal cancer, breast cancer, colon cancer, gastric cancer, liver cancer, leukemia, kidney cancer, lung cancer, intestinal cancer, bone cancer, prostate cancer, cervical cancer, lymphoma, adrenal gland tumor, bladder tumor, or a combination thereof.

In the tenth aspect of the present invention, it provides a method for treating a disease which comprises a step of administering a safe and effective amount of the small protein targeting PD-L1 or the recombinant protein thereof according to the first aspect of the present invention, or the fusion protein according to the second aspect, or the immunoconjugate according to the sixth aspect, or the pharmaceutical composition of the seventh aspect of the present invention, to a subject in need thereof.

It should be understood that, within the scope of the present invention, the technical features specifically described above and below (such as the Examples) can be combined with each other, thereby constituting a new or preferred technical solution which needs not be described one by one.

### DESCRIPTION OF FIGURE

FIG. 1 shows the structural simulation of a complex of the ultrahigh-affinity binding small protein targeting PD-L1 with human PD-L1.
A shows the protein structure of the complex of human PD-1 and PD-L1.
B shows the structural simulation of the complex formed by the binding of the small protein PD-L1-\textcircled{3} with human PD-L1.
C shows the structural simulation of the complex formed by the binding of the small protein PD-L1-\textcircled{1} with human PD-L1.
D shows the structural simulation of the complex formed by the binding of the small protein PD-L1-\textcircled{5} with human PD-L1.
E shows the structural simulation of the complex formed by the binding of the small protein PD-L1-\textcircled{2} with human PD-L1.

FIG. 2 shows the schematic diagram of several structural combinations of high affinity PD-1 small proteins and the fusion proteins thereof.
A shows the short peptide chain of the small protein targeting PD-L1.
B shows a single/multi-targeting fusion protein targeting PD-L1 which is formed utilizing the high affinity PD-1 protein (or fragment thereof) provided in the present invention and by connecting the small protein targeting PD-L1 with an antibody hinge or linker as well as CH2 and CH3 in series to form a polypeptide chain.
C shows a single/multi-targeting fusion protein targeting PD-L1 which is formed utilizing the high affinity PD-1 protein (or fragment thereof) provided in the present invention and by connecting the small protein targeting PD-L1 with an antibody hinge or linker as well as CH3 in series to form a polypeptide chain.
D shows a single/multi-targeting fusion protein targeting PD-L1 which is formed utilizing the high affinity small protein (or fragment thereof) provided in the present invention and by connecting the small protein targeting PD-L1 with an antibody hinge or linker as well as CH3 in series to form a polypeptide chain.
E shows a single/multi-targeting fusion protein targeting PD-L1 which is formed utilizing the high affinity PD-1 protein (or fragment thereof) provided in the present invention and by connecting the small protein targeting PD-L1 to another small protein targeting PD-L1 with a linker sequence, then connecting it to an antibody hinge or linker as well as CH2 and CH3 in series to form a polypeptide chain.
F shows a single/multi-targeting fusion protein targeting PD-L1 which is formed utilizing the high affinity small protein targeting PD-L1 (or fragment thereof) provided in the present invention and by connecting the small protein targeting PD-L1 to another small protein targeting PD-L1 with a linker sequence, then connecting it to an antibody hinge or linker as well as CH3 in series to form a polypeptide chain.
G shows a single/multi-targeting fusion protein targeting PD-L1 which is formed utilizing the high affinity small protein targeting PD-L1 (or fragment thereof) provided in the present invention and by connecting the small protein targeting PD-L1 to another small protein targeting PD-L1 with a linker sequence, then connecting it to an antibody hinge or linker as well as CH3 in series to form a polypeptide chain.

FIG. 3 shows the binding activity of the ultrahigh-affinity small protein targeting PD-L1 detected by flow cytometry.

The ultrahigh-affinity small protein targeting PD-L1 is displayed on the surface of yeast, and the yeast displaying the small protein is traced using anti-Myc tag antibody FITC (ab1394). Yeast cells capable of binding to biotinylated human PD-L1 protein are traced using Avidin, NeutrAvidin™ and PE conjugate (A2660).

FIG. 4 shows the competitive binding activity of the ultrahigh-affinity small protein targeting PD-L1 with wild-type human PD-1 detected by flow cytometry.

Human PD-1 protein at different concentrations is incubated with biotinylated PD-L1 at room temperature, followed by incubation with yeast displaying the ultrahigh-affinity small protein targeting PD-L1. The competitive binding activity of the ultrahigh-affinity small protein targeting PD-L1 with human PD-1 is assessed with flow cytometry using double staining of anti-Myc tag antibody FITC (ab1394) and Avidin, NeutrAvidin™, PE conjugate (A2660).

FIG. 5 shows the affinity of the ultrahigh-affinity small protein targeting PD-L1 for PD-L1 measured using Biolayer Interferometry (BLI).

Biotinylated human PD-L1 is coated on the detection probe, and the affinity of different concentrations of the ultrahigh-affinity small protein targeting PD-L1 for human PD-L1 is detected.

FIG. 6 shows the thermal stability of the ultrahigh-affinity small protein targeting PD-L1 measured using a CD spectrometer.

The protein circular dichroism of PD-L1-3) is observed at three temperature levels: 25° C., heating to 95° C., and cooling back to 25° C., thereby assessing changes in the protein's secondary structure before and after heating.

FIG. 7 shows the Tm value of the ultrahigh-affinity small protein targeting PD-L1 measured using a CD spectrometer.

The circular dichroism signal of PD-L1-3) is monitored as it is gradually heated from 25° C. to 95° C. The Tm value of the protein is calculated based on the time-dependent changes in the protein circular dichroism signal.

### EMBODIMENTS

After extensive and intensive research, the inventors have obtained a class of ultrahigh-affinity small proteins targeting PD-L1 through a large number of screenings based on the structure of wild-type PD-1/PD-L1 proteins and the interaction surface between PD-1 and PD-L1. The binding sites of the small protein can almost completely cover the wild-type PD-1/PD-L1 binding sites. Experiments show that the high-affinity small protein of the present invention has a much higher affinity than the wild-type PD-1 protein. In addition, the small protein of the present invention has a smaller molecular weight as compared to traditional antibodies, having a potentially better tumor penetration. On this basis, the present invention has been completed.

Specifically, the representative ultrahigh-affinity small protein targeting PD-L1 has a length of less than about 60 amino acids, a molecular weight far smaller than conventional antibodies, and no antibody Fc fragment, thus having better tumor penetration. In addition, the ultrahigh-affinity small protein targeting PD-L1 of the present invention has higher affinity and can be used as a potential tumor PD-L1 expression tracing probe.

### Ultrahigh-Affinity Small Protein and Fusion Protein Targeting PD-L1 of the Present Invention

In the present invention, it provides an ultrahigh-affinity small protein targeting PD-L1 and a fusion protein comprised the small protein or an immunoconjugate thereof.

As used herein, the terms “small protein of the present invention” and “ultrahigh-affinity small protein targeting PD-L1 of the present invention” are interchangeably used, and both refer to the small protein that has an ultrahigh affinity for human PD-L1 according to the first aspect of the present invention.

Preferably, the small protein of the present invention has an amino acid sequence set forth in SEQ ID NOs: 1, 3, 5 or 7.

As used herein, the term “fusion protein of the present invention” refers to a fusion protein formed by the ultrahigh-affinity small protein targeting PD-L1 of the present invention and other fusion elements. For example, the small protein of the present invention can form a fusion protein with an element such as a hinge region, an Fc region, and the like. The fusion protein of the present invention has an ultrahigh affinity for PD-L1.

As used herein, the term “having an ultrahigh affinity for PD-L1” indicates that the affinity of the small protein or fusion protein of the present invention for wild-type human PD-L1 protein is much higher than that of wild-type PD-1 protein for wild-type human PD-L1 protein. For example, the affinity of the small protein or fusion protein of the present invention for wild-type human PD-L1 protein (Q1) is at least 1.5 times, at least 2 times or more than the affinity of wild-type PD-1 protein for wild-type human PD-L1 protein (Q0); or the ratio of the Kd value of the small protein or fusion protein of the present invention (Z1) for wild-type human PD-L1 protein to the Kd value of wild-type PD-1 protein (Z0) for wild-type human PD-L1 protein (Z1/Z0) is ≤1/1.5, preferably ≤1/2 or ≤1/3 or less. Preferably, the ultrahigh-affinity fusion protein of the present invention can be any fusion protein that at least comprises a complete ultrahigh-affinity small protein targeting PD-L1 or a partial amino acid fragment thereof (typically an amino acid fragment of at least 70% length).

Typically, the fusion protein of the present invention may have the following structures:
A Y-shaped structure of ultrahigh-affinity small protein or fragment targeting PD-L1-Hinge-CH2-CH3;
A Y-shaped structure of ultrahigh-affinity small protein or fragment targeting PD-L1-Hinge-CH3;
Ultrahigh-affinity small protein or fragment targeting PD-L1-tracer; or
Ultrahigh-affinity small protein or fragment targeting PD-L1.

It should be understood that the above structural types are only exemplary and do not limit the present invention. Some representative structures are shown in FIG. 2. Among them, the ultrahigh-affinity small protein targeting PD-L1 or the fragment thereof can be a single one or multiple ones (such as 2, 3, or 4 ultrahigh-affinity small proteins or the fragments in a tandem form, such as shown in FIGS. 2E, 2F, and 2G).

As used herein, the terms “ultrahigh-affinity small protein targeting PD-L1” or “fusion protein” also include variant forms that have PD-L1 binding activity as well as PD-1/PD-L1 blocking activity. These variant forms include (but are not limited to): deletion, insertion and/or substitution of 1-3 (usually 1-2, more preferably 1) amino acids, addition or deletion of one or several (usually within 3, preferably within 2, more preferably within 1) amino acids at the C-terminus and/or N-terminus, or addition of an amino acid fragment with a relatively small amino acid side chain to the N-terminus or C-terminus of the small protein as a linker (e.g., glycine, serine, etc.). For example, in this art, substitution with amino acids with similar or close properties usually does not alter the function of a protein. For another example, addition or deletion of one or several amino acids at the C-terminus and/or N-terminus usually also does not alter the structure and function of a protein. In addition, the term also includes the polypeptides of the present invention in monomeric and multimeric forms. The term also includes linear and non-linear polypeptides (such as cyclic peptides).

The present invention also includes the active fragments, derivatives and analogs of the above-mentioned ultrahigh-affinity small protein targeting PD-L1 or fusion protein (especially a fusion protein formed by fusing with Fc fragment). As used herein, the terms “fragment”, “derivative” and “analog” refer to a polypeptide that substantially retains the function or activity of ultrahigh-affinity small protein targeting PD-L1 or fusion protein of the present invention.

The polypeptide fragment, derivative or analog of the present invention can be (i) a polypeptide with substitution of one or several conservative or non-conservative amino acid residues (preferably conservative amino acid residues), or (ii) a polypeptide with a substitution group in one or more amino acid residues, or (iii) a polypeptide formed by fusion of the polypeptide with another compound (such as a compound that extends the half-life of the polypeptide, such as polyethylene glycol), or (iv) a polypeptide formed by fusion of an additional amino acid sequence with this polypeptide sequence (a fusion protein formed by fusion with a leader sequence, a secretion sequence, or a tag sequence such as 6His). According to the teachings herein, these fragments, derivatives and analogs belong to the scope well known to those skilled in the art.

A preferred type of the active derivative refers to a polypeptide formed by substitution of at most 3, preferably at most 2, and more preferably at most 1 amino acids with an amino acid having similar or close properties, compared with the amino acid sequence of the present invention. These conservative variant polypeptides are preferably produced by the amino acid substitution according to Table A.

TABLE 1

| | | Preferred |
|---|---|---|
| Initial residue | Representative substitution | substitution |
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

Analogs of the fusion protein of the present invention are also provided in the present invention. The difference between these analogs and the polypeptide of the present invention may be the difference in amino acid sequences, the difference in modification form that does not affect the sequence, or both. The analog also includes an analog with residues different from natural L-amino acids (such as D-amino acids), and an analog with non-naturally occurring or synthetic amino acids (such as β,γ-amino acids). It should be understood that the polypeptide of the present invention is not limited to the representative polypeptide exemplified above.

In addition, the ultrahigh-affinity small protein targeting PD-L1 or fusion protein of the present invention can also be modified. Modification (usually without altering the primary structure) forms include: a chemically derived form of a polypeptide in vivo or in vitro, such as acetylation or carboxylation. The modification also includes glycosylation, such as those polypeptides produced by glycosylation modification during the synthesis and processing of the polypeptide or during further processing steps. This modification can be accomplished by exposing the polypeptide to an enzyme for glycosylation (such as a mammalian glycosylase or deglycosylase). Modification forms also include a sequence with phosphorylated amino acid residues (e.g., phosphotyrosine, phosphoserine, phosphothreonine). A polypeptide that has been modified thereby improving its resistance to proteolysis or optimizing its solubility, is also included.

The term “polynucleotide of the present invention” may include a polynucleotide encoding the ultrahigh-affinity small protein targeting PD-L1 or fusion protein of the present invention, or may also include a polynucleotide containing an additional encoding and/or non-encoding sequence.

A variant of the aforementioned polynucleotide, which encodes a fragment, analog and derivative of the polypeptide or fusion protein with the same amino acid sequence as the present invention, is also related to the present invention. These nucleotide variants include substitution variants, deletion variants and insertion variants. As known in the art, an allelic variant is an alternative form of a polynucleotide, which may be a substitution, deletion or insertion of one or more nucleotides, but will not substantially alter the function of the ultrahigh-affinity small protein targeting PD-L1 or fusion protein it encodes.

A polynucleotide that hybridizes with the aforementioned sequence and has at least 50%, preferably at least 70%, and more preferably at least 80% identity between the two sequences, is also related to the present invention. A polynucleotide that can hybridize with the polynucleotide of the present invention under strict conditions (or stringent conditions) is particularly related to the present invention. In the present invention, “strict conditions” refer to: (1) hybridization and elution at lower ionic strength and higher temperature, such as 0.2×SSC, 0.1% SDS, 60° C.; or (2) a denaturant is added during hybridization, such as 50% (v/v) formamide, 0.1% calf serum/0.1% Ficoll, 42° C., etc.; or (3) the hybridization occurs only when the identity between two sequences is at least more than 90%, preferably more than 95%.

The ultrahigh-affinity small protein targeting PD-L1 or fusion protein and polynucleotide of the present invention are preferably provided in an isolated form, and more preferably, are purified to homogeneity.

The full-length sequence of the polynucleotide of the present invention can usually be obtained by PCR amplification method, recombination method or artificial synthesis method. For the PCR amplification method, primers can be designed according to the relevant nucleotide sequence disclosed in the present invention, especially the open reading frame sequence, and a commercially available cDNA library or a cDNA library prepared by a conventional method known to those skilled in the art can be used as a template to amplify the relevant sequence. When the sequence is long, two or more PCR amplifications are often necessary to be performed, and then the amplified fragments are spliced together in the correct order.

Once a relevant sequence is obtained, recombination methods can be used to obtain the relevant sequence in large quantities. This is usually carried out by cloning the sequence into a vector, transforming a cell with the vector, and then separating the relevant sequence from the proliferated host cell by conventional methods.

In addition, a relevant sequence can be synthesized artificially, especially when the fragment is short in length. Usually, several small fragments are synthesized first, and then are linked together to obtain a fragment with a long sequence.

At present, a DNA sequence encoding the protein (or fragment or derivative thereof) of the present invention can be obtained completely through chemical synthesis. The DNA sequence can then be introduced into various existing DNA molecules (or such as vectors) and cells known in the art.

The method of using PCR technology to amplify DNA/RNA is preferably used to obtain the polynucleotide of the present invention. Especially when it is difficult to obtain full-length cDNA from the library, the RACE method (RACE-rapid amplification of cDNA ends) can be preferably used. The primers used for PCR can be appropriately selected according to the sequence information of the present invention disclosed herein, and can be synthesized by conventional methods. The amplified DNA/RNA fragments can be separated and purified by conventional methods such as gel electrophoresis.

### Expression Vector

The present invention also relates to a vector comprising the polynucleotide of the present invention, and a host cell produced by genetic engineering using the vector of the present invention or an encoding sequence of the ultrahigh-affinity small protein targeting PD-L1 or fusion protein of the present invention, and a method for producing the polypeptide of the present invention through recombinant technology.

Through conventional recombinant DNA technology, the polynucleotide sequence of the present invention can be used to express or produce a recombinant fusion protein. Generally, the following steps are included:
(1) transforming or transducing a suitable host cell by using the polynucleotide (or variant) of the present invention encoding the fusion protein of the present invention, or using a recombinant expression vector comprising the polynucleotide;
(2) culturing the host cell in a suitable medium;
(3) separating and purifying the protein from the culture medium or cells.

In the present invention, the polynucleotide sequence encoding the fusion protein can be inserted into the recombinant expression vector. The term “recombinant expression vector” refers to bacterial plasmids, bacteriophages, yeast plasmids, plant cell viruses, and mammalian cell viruses such as adenovirus, retrovirus or other vectors well known in the art. Any plasmid and vector can be used as long as it can be replicated and stabilized in the host. An important feature of an expression vector is that it usually comprises an origin of replication, a promoter, a marker gene, and a translation control element.

The preparation method of the ultrahigh-affinity small protein targeting PD-L1 or the fusion protein thereof of the present invention may employ any suitable vector which may be selected from one of pET, pDR1, pcDNA3.1 (+), pcDNA3.1/ZEO (+), pDHFR. The expression vector includes a fused DNA sequence connected with suitable transcription and translation regulatory sequences.

Both eukaryotic and prokaryotic host cells can be used for the expression of the ultrahigh-affinity small protein targeting PD-L1 or the fusion protein thereof of the present invention. Eukaryotic host cells are preferably mammalian or insect host cell culture systems, wherein COS, CHO, NS0, sf9, and sf21 cells are preferred. The preferred prokaryotic host cell is one of DH5a, BL21 (DE3), and TG1.

Methods well-known to those skilled in the art can be used to construct an expression vector comprising a DNA encoding sequence of the fusion protein of the present invention and an appropriate transcription/translation control signal. These methods include in vitro recombinant DNA technology, DNA synthesis technology, and in vivo recombination technology, etc. The DNA sequence can be effectively linked to an appropriate promoter in the expression vector to guide mRNA synthesis. Representative examples of these promoters are: lac or trp promoter of Escherichia coli; λ phage PL promoter; eukaryotic promoters including CMV immediate early promoter, HSV thymidine kinase promoter, early and late SV40 promoter, LTRs of retroviruses and some other known promoters which can control the gene expression in prokaryotic or eukaryotic cells or viruses. The expression vector also includes a ribosome binding site for translation initiation and a transcription terminator.

In addition, the expression vector preferably comprises one or more selectable marker genes to provide phenotypic traits for selection of transformed host cells, such as dihydrofolate reductase, neomycin resistance, and green fluorescent protein (GFP) for eukaryotic cell culture, or tetracycline or ampicillin resistance for Escherichia coli.

A vector comprising the above-mentioned appropriate DNA sequence and an appropriate promoter or control sequence can be used to transform an appropriate host cell so that it can express a protein.

The host cell can be a prokaryotic cell, such as a bacterial cell; or a lower eukaryotic cell, such as a yeast cell; or a higher eukaryotic cell, such as a mammalian cell. Representative examples include: Escherichia coli, Streptomyces; bacterial cells of Salmonella typhimurium; fungal cells such as yeast and plant cells (such as ginseng cells).

When the polynucleotide of the present invention is expressed in higher eukaryotic cells, the transcription will be enhanced if an enhancer sequence is inserted into the vector. Enhancers are cis-acting factors of DNA, usually with about 10 to 300 base pairs, acting on promoters to enhance gene transcription. Examples that can be enumerated include the SV40 enhancer with 100 to 270 base pairs on the late side of the replication origin, the polyoma enhancer on the late side of the replication origin, and the adenovirus enhancer, etc.

Those of ordinary skill in the art know how to select appropriate vectors, promoters, enhancers and host cells.

Transformation of a host cell with a recombinant DNA can be carried out by conventional techniques well known to those skilled in the art. When the host is a prokaryotic organism such as Escherichia coli, competent cells that can absorb DNA can be harvested after the exponential growth phase then treated with the CaCl₂) method, and the steps used are well known in the art. Another method is to use MgCl₂. If necessary, the transformation can also be carried out by electroporation. When the host is a eukaryote, the following DNA transfection methods can be selected: calcium phosphate co-precipitation method, conventional mechanical methods such as microinjection, electroporation, liposome packaging, etc.

The obtained transformants can be cultured by conventional methods to express the polypeptide encoded by the gene of the present invention. Depending on the host cell used, the medium used during the culture can be selected from various conventional mediums. The culture is carried out under conditions suitable for the growth of the host cell. When the host cell has grown to an appropriate cell density, a suitable method (such as temperature conversion or chemical induction) is used to induce the selected promoter, and the cell is cultured for another period of time.

The recombinant polypeptide in the above method can be expressed intracellularly or on the cell membrane, or be secreted out of the cell. If desired, recombinant proteins can be isolated and purified by various separation methods using their physical, chemical and other properties. These methods are well known to those skilled in the art. Examples of such methods include, but are not limited to, conventional renaturation treatments, treatment with a protein precipitant (salting-out method), centrifugation, osmosis cell disruption, super-treatment, ultracentrifugation, molecular sieve chromatography (gel filtration), adsorption chromatography, ion-exchange chromatography, high performance liquid chromatography (HPLC) and various other liquid chromatography techniques and combinations of these methods.

The separation and purification of the ultrahigh-affinity small protein targeting PD-L1 or the fusion protein thereof disclosed in the present invention can be achieved using affinity chromatography. Depending on the characteristics of the affinity column used, conventional methods such as high-salt buffers or pH changes can be employed to elute the ultrahigh-affinity small protein targeting PD-L1 or the fusion protein thereof bound to the affinity column.

Using the above methods, the ultrahigh-affinity small protein targeting PD-L1 or the fusion protein thereof can be purified to substantially homogeneous, for example, exhibiting a single band on SDS-PAGE electrophoresis.

### Pharmaceutical Composition

The present invention also provides a pharmaceutical composition comprising the small protein targeting PD-L1, or the fusion protein, or the immunoconjugate thereof of the present invention.

The pharmaceutical composition according to the present invention comprises a safe and effective amount (e.g. 0.001-99 wt %, preferably 0.01-90 wt %, preferably 0.1-80 wt %) of the small protein or fusion protein according to the present invention (or a conjugate thereof) and a pharmaceutically acceptable carrier or excipient. Such carriers include (but are not limited to): saline, buffers, glucose, water, glycerol, ethanol, and a combination thereof. Pharmaceutical preparations should correspond to the administration modes. The pharmaceutical composition according to the present invention can be prepared in the form of an injection, for example, by a conventional method using physiological saline or an aqueous solution containing glucose and other adjuvants. A pharmaceutical composition, for example, an injection and a solution, should be prepared under aseptic conditions. The administration amount of an active ingredient is a therapeutically effective amount, for example, about 10 μg per kilogram of body weight to about 50 mg per kilogram of body weight daily. In addition, the polypeptide according to the present invention may also be used in combination with an additional therapeutic agent. The small protein targeting PD-L1, the fusion protein or the immunoconjugate can form a pharmaceutical formulation together with a pharmaceutically acceptable ingredient to exert a more stable therapeutic effect. These formulations can ensure the structural integrity of the core amino acid sequence of the small protein targeting PD-L1 or the fusion protein thereof in the present invention, and at the same time ensure the multiple functional groups of the protein protected against degradation (including but not limited to coagulation, deamination or oxidation). The formulation may be in various forms. Generally, liquid formulations are typically stable for at least one year at 2° C. to 8° C. and lyophilized formulations are stable for at least six months at 30° C. The formulations herein may be suspension, aqueous injection solution, or lyophilized formulation, etc., which are commonly used in the pharmaceutical field, wherein aqueous solution or lyophilized formulation is preferred.

For the pharmaceutical composition of the present invention that targets PD-L1, such as aqueous injection solution or lyophilized formulation, the pharmaceutically acceptable ingredient therein includes one of surfactants, solution stabilizers, isotonicity adjusting agents and buffer solutions, or a combination thereof, wherein the surfactants include nonionic surfactants such as polyoxyethylene sorbitan fatty acid esters (Tween 20 or 80); poloxamer (such as poloxamer 188); Triton; sodium dodecyl sulfate (SDS); sodium lauryl sulfate; tetradecyl, linoleyl or octadecyl sarcosine; Pluronics; MONAQUAT™, etc., the addition amount of which should minimize the granulation tendency of the protein. The solution stabilizer may be sugars including reducing sugar and non-reducing sugaror, amino acids including monosodium glutamate or histidine, or alcohols including one of a trihydroxy alcohol, a higher sugar alcohol, a propylene glycol, and a polyethylene glycol or a combination thereof. The solution stabilizer is added in an amount such that the final formed formulation remains stable for a period of time that is considered stable by those skilled in the art. The isoosmotic adjusting agent may be one of sodium chloride and mannitol, and the buffer may be one of TRIS, histidine buffer, and phosphate buffer.

When a pharmaceutical composition is used, a safe and effective amount of the small protein or the fusion protein or the immunoconjugate thereof of the present invention is administered to a mammal, wherein the safe and effective amount is generally at least about 50 μg per kilogram of body weight, and in most cases, no more than about 100 mg per kilogram of body weight, preferably, the amount is from about 100 μg per kilogram of body weight to about 50 mg per kilogram of body weight. Of course, a specific amount should also depend on the factors such as administration route and physical conditions of a patient, which fall into the skills of skilled physicians. Typically and generally, the total dose administered should not exceed a certain range, such as an intravenous dose of 10 to 3000 mg/day/50 kg, preferably 100 to 1000 mg/day/50 kg.

The small protein targeting PD-L1 or the fusion protein thereof in the present invention and a pharmaceutical preparation containing the same can be used as an anti-tumor drug for tumor treatment. The term “anti-tumor drug” as used in the present invention refers to a drug able to inhibit and/or treat tumor, the effect of which may include a delay of symptoms accompanying the development associated with tumor growth and/or a decrease in the severity of these symptoms, and further include a decreased symptom accompanying tumor growth which already exists and the prevention of other symptoms, and also reduce or prevent metastasis.

The aforementioned small protein targeting PD-L1 or the fusion protein thereof and the pharmaceutical preparation thereof can also be administered for the treatment of tumors in combination with other anti-tumor drugs, wherein the anti-tumor drugs used in combination include but not limited to: 1. Cytotoxic drugs (1) Drugs acting on the chemical structure of DNA: alkylating agents such as nitrogen mustards, nitrosoureas, methyl sulfonates; platinum compounds such as cisplatin, carboplatin and Oxaliplatin and the like; mitomycin (MMC); (2) Drugs affecting nucleic acid synthesis: dihydrofolate reductase inhibitors such as methotrexate (MTX) and Alimta, etc.; thymidine synthase inhibitors such as fluorouracil (5FU, FT-207, capecitabine), etc.; purine nucleoside synthase inhibitors such as 6-mercaptopurine (6-MP) and 6-TG, etc.; nucleoside reductase inhibitors such as hydroxyurea (HU), etc.; DNA polymerase inhibitors such as cytarabine (Ara-C) and Gemz, etc.; (3) Drugs that act on nucleic acid transcription: drugs that selectively act on DNA templates and inhibit DNA-dependent RNA polymerase, thereby inhibiting RNA synthesis, such as actinomycin D, daunorubicin, doxorubicin, epirubicin, aclarubicin, mithramycin, etc.; (4) Drugs mainly acting on tubulin synthesis: paclitaxel, taxotere, vinblastine, vinorelbine, podophyllum, homoharringtonine; (5) Other cytotoxic drugs: Asparaginase mainly inhibiting protein synthesis; 2. Hormone: Anti-estrogen: tamoxifen, droloxifene, exemestane, etc.; Aromatase inhibitors: aminoglutethimide, lentaron, letrozole, anastrozole, etc.; Anti-androgen: flutamide; RH-LH agonist/antagonist: zoladex, enatone, etc.; 3. Biological response modifier: tumor interferon is inhibited mainly through the body's immune function; interleukin-2; thymosin; 4. monoclonal antibodies: Rituximab (Mab Thera); Cetuximab (C225); Herceptin (Trastuzumab); Bevacizumab (Avastin); Yervoy (Ipilimumab); Nivolumab (OPDIVO); Pembrolizumab (Keytruda); Atezolizumab (Tecentriq); 5. Other drugs that the mechanisms currently unknown and need to be further studied: cell differentiation inducers such as retinoids; apoptosis inducers.

The main advantages of the present invention include:
1) The binding site of the small protein targeting PD-L1 provided by the present invention can cover the binding of wild-type PD-1 to PD-L1.
2) The small protein of the present invention has a smaller molecular weight, with a length of less than approximately 60 amino acids, and possesses better tumor penetration.
3) The small protein of the present invention has an ultrahigh affinity for human PD-L1 which is much higher than the affinity of wild-type PD-1 for PD-L1.
4) The small protein of the present invention exhibits ultrahigh structural stability, with a Tm value greater than 95° C.

The invention is further illustrated below in conjunction with specific embodiments. It should be understood that the examples are not intended to limit the scope of the invention. The experimental methods in the following examples which do not specify the specific conditions are usually in accordance with conventional conditions, such as conditions described in Sambrook et al., Molecular Cloning: Laboratory Manual (New York: Cold Spring Harbor Laboratory Press, 1989), or in accordance with the conditions recommended by the manufacturer. Unless otherwise stated, percentages and parts are by weight.

Sequence of the present invention
Amino acid sequence of PD-L1-\textcircled{3}
(SEQ ID NO: 1)
DRERARELARILLKVIKLSDSPEARRQLLRNLEELAEKYKDPEVRRILEEAERYIK
Nucleotide sequence of PD-L1-\textcircled{3}
(SEQ ID NO: 2)
GACCGTGAACGTGCACGTGAACTGGCTCGCATTCTGCTGAAAGTAATTAAACTGA
GCGACTCCCCAGAAGCACGTCGTCAGCTGCTGCGCAACCTGGAAGAACTGGCGGAAA
AATATAAAGATCCGGAGGTTCGTCGTATCCTGGAAGAAGCCGAACGTTATATCAAA
Amino acid sequence of PD-L1-\textcircled{1}
(SEQ ID NO: 3)
SREAVRQLLEDARKSKDPELVRILLKVARNLAELLNDPELRRLVEEIEEILRRLR
Nucleotide sequence of PD-L1-\textcircled{1}
(SEQ ID NO: 4)
AGCCGTGAAGCAGTACGTCAGCTGCTGGAAGACGCACGTAAATCTAAAGACCCG
GAACTGGTACGCATCCTGCTGAAGGTGGCACGTAACCTGGCGGAGCTGCTGAACGAC
CCAGAACTGCGTCGTCTGGTGGAAGAAATTGAAGAAATCCTGCGCCGTCTGCGT
Amino acid sequence of PD-L1-\textcircled{5}
(SEQ ID NO: 5)
SAREEADRLLQEIARLRKEGDREKAEEIVKRLRELVERLNDPLLRIILKVAENILKELN
Nucleotide sequence of PD-L1-\textcircled{5}
(SEQ ID NO: 6)
TCTGCTCGTGAAGAAGCTGATCGTCTGCTGCAGGAAATCGCTCGTCTGCGCAAGG
AAGGCGATCGTGAAAAAGCAGAAGAAATCGTAAAACGTCTGCGTGAACTGGTTGAAC
GTCTGAACGATCCGCTGCTGCGTATCATCCTGAAAGTTGCTGAAAACATCCTGAAGGA
ACTGAAC
Amino acid sequence of PD-L1-\textcircled{2}
(SEQ ID NO: 7)
SKEEALEQLLRDLKESTDPELIRILLKVIENLARLANNPEYLERAEKIYREL
Nucleotide sequence of PD-L1-\textcircled{2}
(SEQ ID NO: 8)
TCTAAGGAAGAAGCTCTGGAACAGCTGCTGCGCGATCTGAAAGAATCTACCGAT
CCGGAACTGATCCGTATTCTGCTGAAGGTTATTGAAAACCTGGCACGCCTGGCAAATA
ACCCGGAATACCTGGAACGTGCGGAAAAAATTTACCGTGAACTG
Amino acid sequence of hinge region
(SEQ ID NO: 9)
EPKSGDKTHTCPPCP
Nucleotide sequence of hinge region
(SEQ ID NO: 10)
GAGCCCAAATCTGGTGACAAAACTCACACATGCCCACCGTGCCCA
Amino acid sequence of Fc
(SEQ ID NO: 11)
APELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLDSDGSFFLYS
KLTVDKSRWQQGNVFSCSVMHEALHNHYTQKSLSLSPGK
Nucleotide sequence of Fc
(SEQ ID NO: 12)
GCACCTGAACTCCTGGGGGGACCGTCAGTCTTCCTCTTCCCCCCAAAACCCAAGG
ACACCCTCATGATCTCCCGGACCCCTGAGGTCACATGCGTGGTGGTGGACGTGAGCCA
CGAAGACCCTGAGGTCAAGTTCAACTGGTACGTGGACGGCGTGGAGGTGCATAATGC
CAAGACAAAGCCGCGGGAGGAGCAGTACAACAGCACGTACCGTGTGGTCAGCGTCCT
CACCGTCCTGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGGCAGCCCCG
AGAACCACAGGTGTACACCCTGCCCCCATCCCGGGATGAGCTGACCAAGAACCAGGT
CAGCCTGACCTGCCTGGTCAAAGGCTTCTATCCCAGCGACATCGCCGTGGAGTGGGAG
AGCAATGGGCAGCCGGAGAACAACTACAAGACCACGCCTCCCGTGCTGGACTCCGAC
GGCTCCTTCTTCCTCTACAGCAAGCTCACCGTGGACAAGAGCAGGTGGCAGCAGGGGA
ACGTCTTCTCATGCTCCGTGATGCATGAGGCTCTGCACAACCACTACACGCAGAAGAG
CCTCTCCCTGTCTCCGGGTAAA
Amino acid sequence of PD-L1-\textcircled{3}-hinge-CH2-CH3
(SEQ ID NO: 13)
DRERARELARILLKVIKLSDSPEARRQLLRNLEELAEKYKDPEVRRILEEAERYIK
EPKSGDKTHTCPPCP
APELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLDSDGSFFLYS
KLTVDKSRWQQGNVFSCSVMHEALHNHYTQKSLSLSPGK
Nucleotide sequence of PD-L1-\textcircled{3}-hinge-CH2-CH3
(SEQ ID NO: 14)
GACCGTGAACGTGCACGTGAACTGGCTCGCATTCTGCTGAAAGTAATTAAACTGA
GCGACTCCCCAGAAGCACGTCGTCAGCTGCTGCGCAACCTGGAAGAACTGGCGGAAA
AATATAAAGATCCGGAGGTTCGTCGTATCCTGGAAGAAGCCGAACGTTATATCAAA
GAGCCCAAATCTGGTGACAAAACTCACACATGCCCACCGTGCCCAGCACCTGAA
CTCCTGGGGGGACCGTCAGTCTTCCTCTTCCCCCCAAAACCCAAGGACACCCTCATGA
TCTCCCGGACCCCTGAGGTCACATGCGTGGTGGTGGACGTGAGCCACGAAGACCCTGA
GGTCAAGTTCAACTGGTACGTGGACGGCGTGGAGGTGCATAATGCCAAGACAAAGCC
GCGGGAGGAGCAGTACAACAGCACGTACCGTGTGGTCAGCGTCCTCACCGTCCTGCAC
CAGGACTGGCTGAATGGCAAGGAGTACAAGTGCAAGGTCTCCAACAAAGCCCTCCCA
GCCCCCATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTGACCTGCC
TGGTCAAAGGCTTCTATCCCAGCGACATCGCCGTGGAGTGGGAGAGCAATGGGCAGC
CGGAGAACAACTACAAGACCACGCCTCCCGTGCTGGACTCCGACGGCTCCTTCTTCCT
CTACAGCAAGCTCACCGTGGACAAGAGCAGGTGGCAGCAGGGGAACGTCTTCTCATG
CTCCGTGATGCATGAGGCTCTGCACAACCACTACACGCAGAAGAGCCTCTCCCTGTCT
CCGGGTAAA
Amino acid sequence of PD-L1-\textcircled{1}-hinge-CH2-CH3
(SEQ ID NO: 15)
SREAVRQLLEDARKSKDPELVRILLKVARNLAELLNDPELRRLVEEIEEILRRLR
EPKSGDKTHTCPPCP
APELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLDSDGSFFLYS
KLTVDKSRWQQGNVFSCSVMHEALHNHYTQKSLSLSPGK
Nucleotide sequence of PD-L1-\textcircled{1}-hinge-CH2-CH3
(SEQ ID NO: 16)
AGCCGTGAAGCAGTACGTCAGCTGCTGGAAGACGCACGTAAATCTAAAGACCCG
GAACTGGTACGCATCCTGCTGAAGGTGGCACGTAACCTGGCGGAGCTGCTGAACGAC
CCAGAACTGCGTCGTCTGGTGGAAGAAATTGAAGAAATCCTGCGCCGTCTGCGT
GAGCCCAAATCTGGTGACAAAACTCACACATGCCCACCGTGCCCAGCACCTGAA
CTCCTGGGGGGACCGTCAGTCTTCCTCTTCCCCCCAAAACCCAAGGACACCCTCATGA
TCTCCCGGACCCCTGAGGTCACATGCGTGGTGGTGGACGTGAGCCACGAAGACCCTGA
GGTCAAGTTCAACTGGTACGTGGACGGCGTGGAGGTGCATAATGCCAAGACAAAGCC
GCGGGAGGAGCAGTACAACAGCACGTACCGTGTGGTCAGCGTCCTCACCGTCCTGCAC
CAGGACTGGCTGAATGGCAAGGAGTACAAGTGCAAGGTCTCCAACAAAGCCCTCCCA
GCCCCCATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTGACCTGCC
TGGTCAAAGGCTTCTATCCCAGCGACATCGCCGTGGAGTGGGAGAGCAATGGGCAGC
CGGAGAACAACTACAAGACCACGCCTCCCGTGCTGGACTCCGACGGCTCCTTCTTCCT
CTACAGCAAGCTCACCGTGGACAAGAGCAGGTGGCAGCAGGGGAACGTCTTCTCATG
CTCCGTGATGCATGAGGCTCTGCACAACCACTACACGCAGAAGAGCCTCTCCCTGTCT
CCGGGTAAA
Amino acid sequence of PD-L1-\textcircled{5}-hinge-CH2-CH3
(SEQ ID NO: 17)
SAREEADRLLQEIARLRKEGDREKAEEIVKRLRELVERLNDPLLRIILKVAENILKELN
EPKSGDKTHTCPPCP
APELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLDSDGSFFLYS
KLTVDKSRWQQGNVFSCSVMHEALHNHYTQKSLSLSPGK
Nucleotide sequence of PD-L1-\textcircled{5}-hinge-CH2-CH3
(SEQ ID NO: 18)
TCTGCTCGTGAAGAAGCTGATCGTCTGCTGCAGGAAATCGCTCGTCTGCGCAAGG
AAGGCGATCGTGAAAAAGCAGAAGAAATCGTAAAACGTCTGCGTGAACTGGTTGAAC
GTCTGAACGATCCGCTGCTGCGTATCATCCTGAAAGTTGCTGAAAACATCCTGAAGGA
ACTGAAC
GAGCCCAAATCTGGTGACAAAACTCACACATGCCCACCGTGCCCAGCACCTGAA
CTCCTGGGGGGACCGTCAGTCTTCCTCTTCCCCCCAAAACCCAAGGACACCCTCATGA
TCTCCCGGACCCCTGAGGTCACATGCGTGGTGGTGGACGTGAGCCACGAAGACCCTGA
GGTCAAGTTCAACTGGTACGTGGACGGCGTGGAGGTGCATAATGCCAAGACAAAGCC
GCGGGAGGAGCAGTACAACAGCACGTACCGTGTGGTCAGCGTCCTCACCGTCCTGCAC
CAGGACTGGCTGAATGGCAAGGAGTACAAGTGCAAGGTCTCCAACAAAGCCCTCCCA
GCCCCCATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTGACCTGCC
TGGTCAAAGGCTTCTATCCCAGCGACATCGCCGTGGAGTGGGAGAGCAATGGGCAGC
CGGAGAACAACTACAAGACCACGCCTCCCGTGCTGGACTCCGACGGCTCCTTCTTCCT
CTACAGCAAGCTCACCGTGGACAAGAGCAGGTGGCAGCAGGGGAACGTCTTCTCATG
CTCCGTGATGCATGAGGCTCTGCACAACCACTACACGCAGAAGAGCCTCTCCCTGTCT
CCGGGTAAA
Amino acid sequence of PD-L1-\textcircled{2}-hinge-CH2-CH3 (SEQ ID NO: 19)
SKEEALEQLLRDLKESTDPELIRILLKVIENLARLANNPEYLERAEKIYREL
EPKSGDKTHTCPPCP
APELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLDSDGSFFLYS
KLTVDKSRWQQGNVFSCSVMHEALHNHYTQKSLSLSPGK
Nucleotide sequence of PD-L1-\textcircled{2}-hinge-CH2-CH3
(SEQ ID NO: 20)
TCTAAGGAAGAAGCTCTGGAACAGCTGCTGCGCGATCTGAAAGAATCTACCGAT
CCGGAACTGATCCGTATTCTGCTGAAGGTTATTGAAAACCTGGCACGCCTGGCAAATA
ACCCGGAATACCTGGAACGTGCGGAAAAAATTTACCGTGAACTG
GAGCCCAAATCTGGTGACAAAACTCACACATGCCCACCGTGCCCAGCACCTGAA
CTCCTGGGGGGACCGTCAGTCTTCCTCTTCCCCCCAAAACCCAAGGACACCCTCATGA
TCTCCCGGACCCCTGAGGTCACATGCGTGGTGGTGGACGTGAGCCACGAAGACCCTGA
GGTCAAGTTCAACTGGTACGTGGACGGCGTGGAGGTGCATAATGCCAAGACAAAGCC
GCGGGAGGAGCAGTACAACAGCACGTACCGTGTGGTCAGCGTCCTCACCGTCCTGCAC
CAGGACTGGCTGAATGGCAAGGAGTACAAGTGCAAGGTCTCCAACAAAGCCCTCCCA
GCCCCCATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTGACCTGCC
TGGTCAAAGGCTTCTATCCCAGCGACATCGCCGTGGAGTGGGAGAGCAATGGGCAGC
CGGAGAACAACTACAAGACCACGCCTCCCGTGCTGGACTCCGACGGCTCCTTCTTCCT
CTACAGCAAGCTCACCGTGGACAAGAGCAGGTGGCAGCAGGGGAACGTCTTCTCATG
CTCCGTGATGCATGAGGCTCTGCACAACCACTACACGCAGAAGAGCCTCTCCCTGTCT
CCGGGTAAA
Amino acid sequence of signal peptide
(SEQ ID NO: 21)
MGWSCIILFLVATATGVHS
Nucleotide sequence of signal peptide
(SEQ ID NO: 22)
ATGGGATGGTCATGTATCATCCTTTTTCTAGTAGCAACTGCAACCGGTGTACATTC
C

### Example 1: Synthesis of High Affinity Protein Targeting Human PD-1

### 1.1 Screening of High Affinity Protein Targeting Human PD-1

Candidate proteins were screened using yeast display library technology. Firstly, the synthetic candidate protein gene was electrotransformed into EBY-100 yeast cells along with pETCON vector fragments at a ratio of 2:1 by using an electroporation method. After culturing on a double-defective (−Ura/−Trp) plate at 30° C. for 2 days, the electrotransformation efficiency was confirmed (to be greater than 1×10⁵). The electrotransformed yeast cells were cultured in double-defective medium at 30° C., 250 rpm for two days. Then, the yeast cells were diluted at a ratio of 1:100 and induced to express the displayed protein in a lactose-rich induction medium. When OD600 reached 0.5, biotinylated PD-L1 was used as the target protein (PD1-H82E5-200 μg), and double-color flow cytometry staining was performed using Avidin, NeutrAvidin™, PE conjugate (A2660), and anti-Myc tag antibody FITC (ab1394). Among them, FITC-positive cells indicate yeast cells displaying the protein, and PE/FITC double-positivity indicates that the displayed protein has a binding affinity with the target protein PD-L1. Yeast cells with PE/FITC double-positivity corresponding to ultrahigh affinity were selected based on their affinity, and the gene sequences of the candidate proteins that are able to bind the target protein (i.e., PD-L1 ultrahigh-affinity peptide) were obtained through gene sequencing.

### 1.2 Synthesis of High Affinity Protein Targeting Human PD-1

Genes of ultrahigh affinity small proteins targeting PD-L1 were synthesized using whole gene synthesis method, which were named PD-L1-\textcircled{3}, PD-L1-\textcircled{1}), PD-L1-\textcircled{5}) and PD-L1-\textcircled{2}) The amino acid sequence of PD-L1-\textcircled{3} is shown in SEQ ID NO: 1, and the nucleotide sequence thereof is shown in SEQ ID NO: 2. The amino acid sequence of PD-L1-\textcircled{1} is shown in SEQ ID NO: 3, and the nucleotide sequence thereof is shown in SEQ ID NO: 4. The amino acid sequence of PD-L1-\textcircled{5} is shown in SEQ ID NO: 5, and the nucleotide sequence thereof is shown in SEQ ID NO: 6. The amino acid sequence of PD-L1-\textcircled{2} is shown in SEQ ID NO: 7, and the nucleotide sequence thereof is shown in SEQ ID NO: 8. The synthesized nucleotide sequence was added with initiation codon to the N-terminus, and inserted into the pET29b(+) expression vector between the XhoI and NedI restriction sites.

### Example 2: Expression and Purification of Ultrahigh-Affinity Small Protein

The vector was transformed into E. coli and cultured in LB medium at 37° C., 270 rpm until the OD600 reached 0.6. Protein expression was then induced overnight with 1 mM IPTG. After harvesting the bacteria, Protease Inhibitor Cocktail and Benzonase® nuclease were added, and the cells were lysed through ultrasonication (6 minutes, 10 s on, 10 s off, 80% Amp). The supernatant was collected. After purification by a Ni column, the concentrated sample was further purified through a molecular sieve. Protein expression and purification was assessed using SDS-PAGE and Coomassie Blue staining. The protein concentration was further determined using BCA method.

High-purity candidate proteins were obtained through such process for subsequent experiments.

### Example 3: Detection of Binding Activity of High Affinity Small Protein Targeting PD-L1

In this Example, the synthesized nucleotide sequence of the small protein was added with initiation codon to the N-terminus, and inserted into the pETCON vector between the XhoI and NedI restriction sites. The vector inserted with the small protein gene was transformed into EBY-100 yeast cells by using a yeast transformation kit. After culturing on a double-defective (−Ura/−Trp) plate at 30° C. for 2 days, the electrotransformation efficiency was confirmed (to be greater than 1×10⁵). The electrotransformed yeast cells were cultured in double-defective medium at 30° C., 225 rpm for two days. Then, the yeast cells were diluted at a ratio of 1:100 and induced to express the displayed protein in a lactose-rich induction medium. When the OD600 reaches 0.5, the yeast cells were incubated at room temperature for 45 minutes with biotin-labeled PD-L1, which was used as the target protein (PD1-H82E5-200 μg) and diluted to concentrations of 1.44 nM, 144 pM, and 14.4 pM. Double-color flow cytometry staining was performed using Avidin, NeutrAvidin™, PE conjugate (A2660), and anti-Myc tag antibody FITC (ab1394). Among them, FITC-positive cells indicate yeast cells displaying the protein, and PE/FITC double-positivity indicates that the displayed protein is able to bind with the target protein.

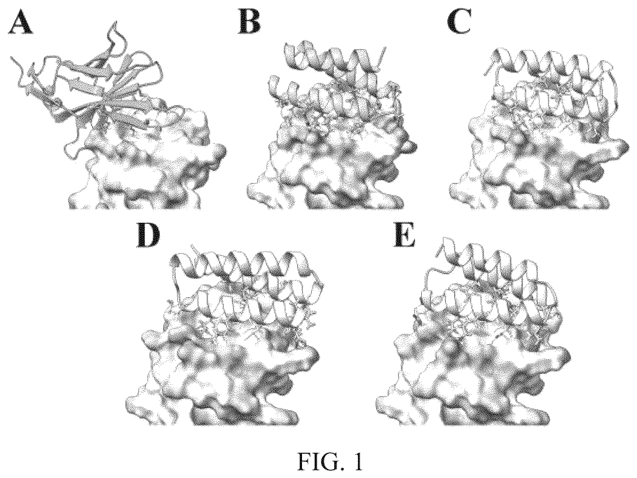

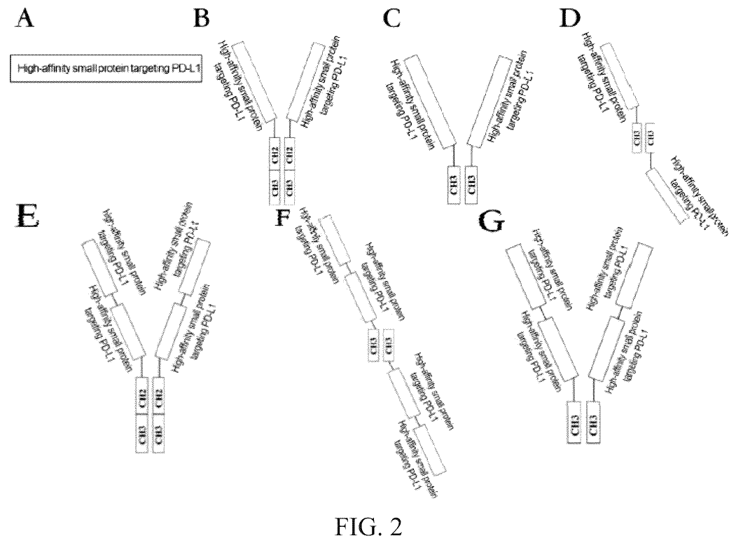

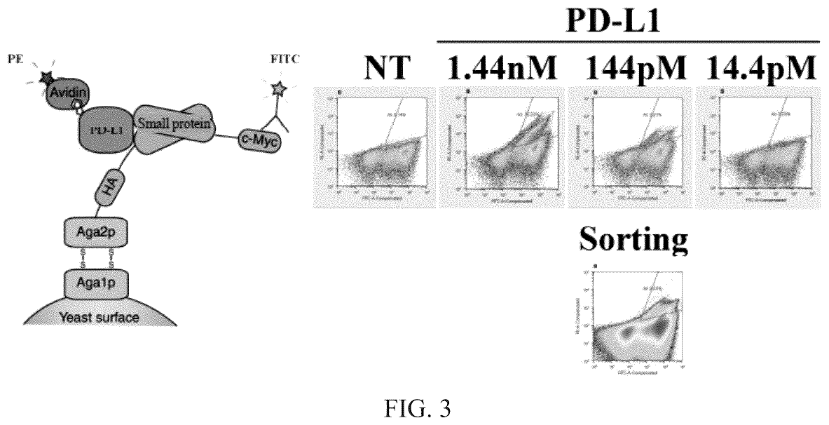

As shown in FIG. 3, the candidate protein displayed on the surface of the yeast cells is able to bind to the target protein at concentrations of 1.44 nM and 144 pM, which exhibits a PE/FITC double-positive signal. By sorting the PE/FITC double-positive yeast cells at a target protein PD-L1 concentration of 144 PM and sequencing them, the gene sequences of the high-affinity candidate protein targeting PD-L1 were obtained.

The binding simulation of the complex structures of human PD-1 and several preferred small proteins of the present invention with human PD-L1 is shown in FIG. 1. Unlike the secondary structure of human PD-1, the peptide chain of the small protein of the present invention mainly comprises three α-helical secondary structures.

### Example 4: Detection of Competitive Binding Activity of High Affinity Small Protein Targeting PD-L1

In this example, to further confirm the competitive binding activity of the high affinity small protein targeting PD-L1 with human PD-1, we firstly incubated PD-1-Fc fusion protein Human PD-1/PDCD1 Protein, Fc Tag (PD1-H5257-100 μg) at different concentrations with biotinylated PD-L1 for 20 minutes at room temperature, then incubated it with yeast cells displaying the high affinity small protein targeting PD-L1. Then, the competitive binding activity was assessed through double-color flow cytometry using Avidin, NeutrAvidin™, PE conjugate (A2660), and anti-Myc tag antibody FITC (ab1394). Among them, FITC-positive cells indicate yeast cells displaying the protein, and PE/FITC double-positivity indicates the binding of the displayed small protein with human PD-L1.

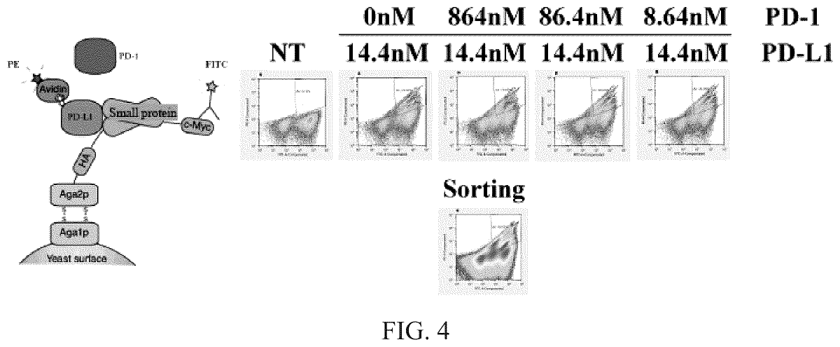

As shown in FIG. 4, with a target protein PD-L1 concentration of 14.4 nM, PD-1 protein concentrations were set at 864 nM, 86.4 nM, 8.64 nM, and 0 nM and incubated with the target protein PD-L1 for 30 minutes at room temperature. Then, the protein incubation mixture was incubated with yeast cells expressing the candidate proteins for 45 minutes at room temperature. The competitive binding activity of the candidate protein was assessed using dual-color flow cytometry. Even at a concentration of 864 nM (a supersaturating concentration) of the competing protein PD-1, the candidate binding protein still demonstrates good competitive protection activity.

### Example 5: Determination of Affinity of High Affinity Small Protein Targeting PD-L1

In this example, the affinity of the high affinity blocking protein was detected using ForteBio Octet. Firstly, 3 μg/ml of biotinylated human PD-L1 protein was loaded onto a detection probe coupled with avidin (300 s), and unbound biotinylated human PD-L1 protein was eluted in PBST solution. Then, the detection probe loaded with human PD-L1 protein was immersed simultaneously in solutions of the high affinity small protein targeting PD-L1 that were two-fold serially diluted, and the binding signal was detected (300 s). Afterward, the probe was immersed in PBST to detect the dissociation signal of the bound protein. Finally, the affinity of the high affinity blocking protein was calculated.

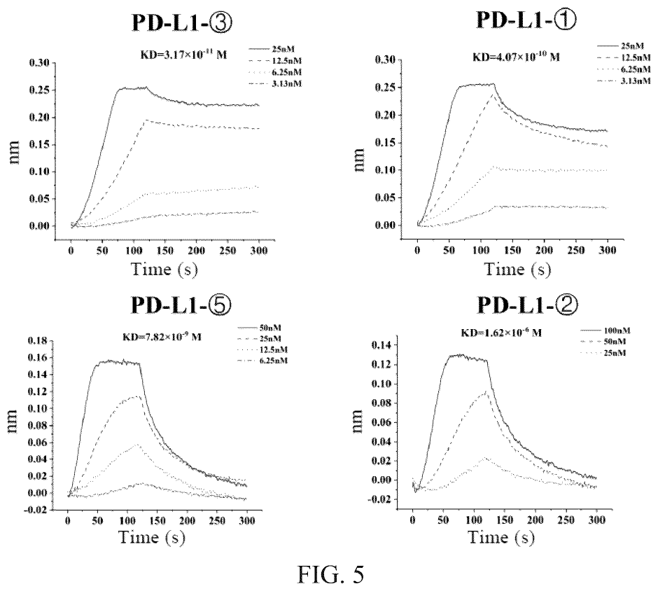

As shown in FIG. 5, PD-L1-\textcircled{3} and PD-L1-\textcircled{1} demonstrated ultrahigh binding activity with affinities of 3.17×10⁻¹¹ M and 4.07×10⁻¹⁰ M, respectively. The affinities of PD-L1-\textcircled{5} and PD-L1-\textcircled{2} were 7.82×10⁻⁹ M and 1.62×10⁻⁶ M, respectively.

### Example 6: Detection of Structural Stability of High Affinity Small Protein Targeting PD-L1

Structural stability of the protein was detected using JASCO-1500. The wavelength range of 190 nm-260 nm was selected for detection. Firstly, the circular dichroism signal of PD-L1-\textcircled{3} protein at 25° C. (0.1 mg/ml) was measured. Then, the protein was heated to 95° C. and the circular dichroism signal was detected. Finally, the temperature was restored to 25° C. and the protein was allowed to stand for 5 minutes before measuring the circular dichroism signal again. The changes in the secondary structure conformation of the protein at different temperatures were obtained to assess the structural stability of the binding protein.

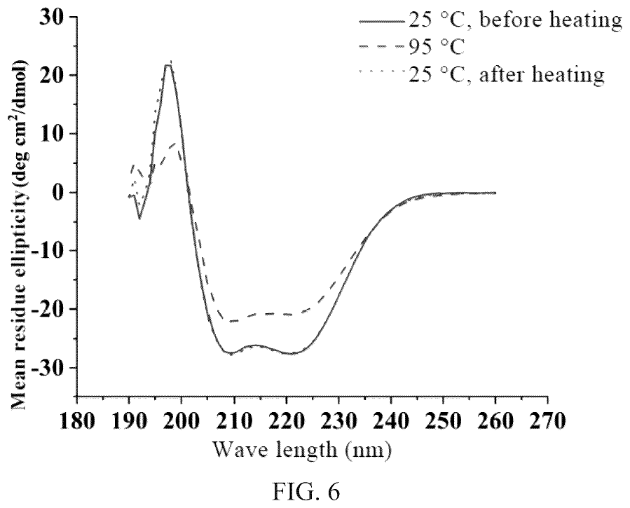

As shown in FIG. 6, PD-L1-\textcircled{3} exhibits a high α-helix secondary structure at 25° C. When heated to 95° C., the secondary structure of the protein shows certain changes due to the high temperature. However, as the temperature is reduced back to 25° C., the circular dichroism signal almost completely overlaps, indicating that the secondary structure of the protein returns to the pre-heating state. The protein exhibits excellent thermal stability.

### Example 7: Measurement of Tm Value of ACE2 High Affinity Binding Blocking Protein

The circular dichroism signal of PD-L1-\textcircled{3} protein at 25° C. (0.1 mg/ml) was determined using JASCO-1500. The circular dichroism signal of the protein as it gradually heated from 25° C. to 95° C. was detected at a selected wavelength of 222 nm. During this process, the heating rate was set at 2° C./minute with a 30-second equilibration period for each minute. The Tm value of the protein was obtained thereby.

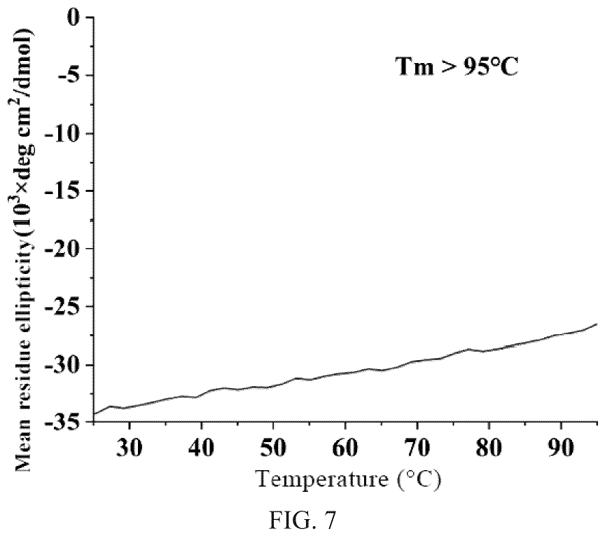

As shown in FIG. 7, although the circular dichroism signal increases as the temperature rises, there is only a slight increase in the signal at 95° C. which is the instrument's detection limit. Based on the signal curve, it was determined that the Tm exceeded the upper temperature detection limit of the instrument, with a Tm greater than 95° C. The protein exhibits excellent thermal stability.

### Example 8: Expression and Purification of Fusion Protein

In this example, fusion proteins of ultrahigh-affinity small proteins are prepared. The structure of the prepared fusion protein is shown in FIG. 2B, and the amino acid sequence is shown in SEQ ID NOs: 13, 15, 17, or 19. The method is as follows:

The encoding sequences of the fusion proteins, SEQ ID NOs: 14, 16, 18, or 20, were introduced into the multiple cloning site of the pcDNA3.1 vector. The vector was transfected into 293F cells and cultured for 6 days in a cell culture incubator. The cell culture supernatant was collected, filtered, and purified using a Protein A column. The sample was then further concentrated using ultrafiltration. Protein expression and purification was assessed using SDS-PAGE and Coomassie Blue staining.

The molecular weights of the various recombinant proteins obtained were detected and found to be consistent with the predicted molecular weights.

In addition, the binding of the fusion protein to PD-L1 was determined using the method described in Example 5. The results show that the prepared fusion proteins are able to bind to PD-L1 with ultrahigh affinity.

All documents mentioned in the present invention are incorporated by reference herein as if each document were incorporated separately by reference. Furthermore, it should be understood that after reading the foregoing teachings of the invention, various changes or modifications may be made to the invention by those skilled in the art and that these equivalents are equally within the scope of the claims appended to this application.

1. A small protein targeting PD-L1, wherein the small protein specifically targets PD-L1 with an ultrahigh affinity, and competes with wild-type PD-1 for binding to PD-L1 and effectively blocks binding of PD-1 to PD-L1;
wherein, the small protein consists of a peptide chain which mainly forms three α-helix secondary structures; and
the amino acid sequence of the small protein is shown in SEQ ID NOs: 1, 3, 5 or 7.

2. A recombinant protein comprising two or more small proteins targeting PD-L1 according to claim 1 in tandem.

3. A fusion protein which comprises a first polypeptide and/or a second polypeptide;
wherein, the first polypeptide has a structure as shown in Formula I from N-terminus to C-terminus, and the second polypeptide has a structure as shown in Formula II from N-terminus to C-terminus, 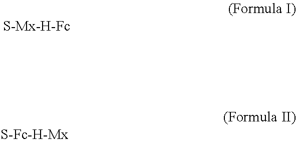
wherein,
S is absent or a signal peptide sequence;
M is a PD-L1 binding domain (or binding element), the amino acid sequence of which is derived from the amino acid sequence of the small protein targeting PD-L1 according to claim 1;
H is a hinge region;
Fc is absent or a constant region of an immunoglobulin, or a fragment thereof;
“-” is a peptide bond or a linking peptide connecting the above elements;
X is a positive integer selected from 1-4.

4. The fusion protein according to claim 3, wherein the fusion protein is a monomer, or a dimer selected from a group consisting of: a homodimer formed by two first polypeptides, a homodimer formed by two second polypeptides, or a heterodimer formed by the first polypeptide and the second polypeptide.

5. The fusion protein according to claim 3, wherein the fusion protein is a homodimer formed by two first polypeptides.

6. The fusion protein according to claim 4, wherein a disulfide bond can be formed between a first polypeptide and another first polypeptide, between a second peptide and another second polypeptide, or between a first polypeptide and a second polypeptide, through cysteines (C) on their Fc region respectively.

7. The fusion protein according to claim 3, wherein the amino acid sequence of the first polypeptide is selected from a group consisting of:
(i) a sequence as shown in SEQ ID NOs: 13, 15, 17 or 19; and
(ii) an amino acid sequence obtained by replacing, deleting, altering or inserting one or more amino acid residues, or adding 1-30 amino acid residues, preferably 1-10 amino acid residues, more preferably 1-5 amino acid residues on the basis of SEQ ID NOs: 13, 15, 17 or 19.

8. A polynucleotide encoding the small protein targeting PD-L1 according to claim 1, or a recombinant protein comprising two or more small proteins targeting PD-L1 according to claim 1 in tandem.

9. A vector comprising the polynucleotide according to claim 8.

10. A host cell, wherein the host cell comprises the vector according to claim 9.

11. An immunoconjugate which comprises:
(a) the small protein targeting PD-L1 according to claim 1, or a recombinant protein comprising two or more small proteins targeting PD-L1 according to claim 1 in tandem; and
(b) a coupling moiety selected from a group consisting of a detectable marker, a drug, a toxin, a cytokine, a radionuclide, or an enzyme.

12. A pharmaceutical composition which comprises:
(a) the small protein targeting PD-L1 according to claim 1, or a recombinant protein comprising two or more small proteins targeting PD-L1 according to claim 1 in tandem, or an encoding gene thereof; and
(b) a pharmaceutically acceptable carrier.

13-14. (canceled)

15. A method for treating a disease which comprises a step of administering a safe and effective amount of the small protein targeting PD-L1 according to claim 1, or a recombinant protein comprising two or more small proteins targeting PD-L1 according to claim 1 in tandem, or a pharmaceutical composition comprising the small protein or the recombinant protein or an encoding gene thereof, to a subject in need thereof.

16. A small protein targeting PD-L1, wherein the small protein specifically targets PD-L1 with an ultrahigh affinity, and competes with wild-type PD-1 for binding to PD-L1 and effectively blocks binding of PD-1 to PD-L1; wherein, the small protein consists of a peptide chain which mainly forms three α-helix secondary structures;
and the amino acid sequence of the small protein is shown in SEQ ID NO: 1.

17-19. (canceled)

20. A polynucleotide encoding the fusion protein according to claim 3.

21. A pharmaceutical composition which comprises:
(a) the fusion protein according to claim 3, or an encoding gene thereof; and
(b) a pharmaceutically acceptable carrier.

22. A method for treating a disease which comprises a step of administering a safe and effective amount of the fusion protein according to claim 3, or a pharmaceutical composition comprising the fusion protein or an encoding gene thereof, to a subject in need thereof.


SEQUENCE LISTING (22 sequences)

SEQ ID NO: 1 - protein, 56 aa, synthetic construct
  source 1..56: note = Small protein PD-L1-3
DRERARELARILLKVIKLSDSPEARRQLLRNLEELAEKYKDPEVRRILEEAERYIK

SEQ ID NO: 2 - DNA, 168 bp, synthetic construct
  source 1..168: mol_type = other DNA; note = Small protein PD-L1-3
gaccgtgaacgtgcacgtgaactggctcgcattctgctgaaagtaattaaactgagcgactccccagaagcacgtcgtcagctgctgcgcaacctggaagaactggcggaaaaatataaagatccggaggttcgtcgtatcctggaagaagccgaacgttatatcaaa

SEQ ID NO: 3 - protein, 55 aa, synthetic construct
  source 1..55: note = Small protein PD-L1-1
SREAVRQLLEDARKSKDPELVRILLKVARNLAELLNDPELRRLVEEIEEILRRLR

SEQ ID NO: 4 - DNA, 165 bp, synthetic construct
  source 1..165: mol_type = other DNA; note = Small protein PD-L1-1
agccgtgaagcagtacgtcagctgctggaagacgcacgtaaatctaaagacccggaactggtacgcatcctgctgaaggtggcacgtaacctggcggagctgctgaacgacccagaactgcgtcgtctggtggaagaaattgaagaaatcctgcgccgtctgcgt

SEQ ID NO: 5 - protein, 59 aa, synthetic construct
  source 1..59: note = Small protein PD-L1-5
SAREEADRLLQEIARLRKEGDREKAEEIVKRLRELVERLNDPLLRIILKVAENILKELN

SEQ ID NO: 6 - DNA, 177 bp, synthetic construct
  source 1..177: mol_type = other DNA; note = Small protein PD-L1-5
tctgctcgtgaagaagctgatcgtctgctgcaggaaatcgctcgtctgcgcaaggaaggcgatcgtgaaaaagcagaagaaatcgtaaaacgtctgcgtgaactggttgaacgtctgaacgatccgctgctgcgtatcatcctgaaagttgctgaaaacatcctgaaggaactgaac

SEQ ID NO: 7 - protein, 52 aa, synthetic construct
  source 1..52: note = Small protein PD-L1-2
SKEEALEQLLRDLKESTDPELIRILLKVIENLARLANNPEYLERAEKIYREL

SEQ ID NO: 8 - DNA, 156 bp, synthetic construct
  source 1..156: mol_type = other DNA; note = Small protein PD-L1-2
tctaaggaagaagctctggaacagctgctgcgcgatctgaaagaatctaccgatccggaactgatccgtattctgctgaaggttattgaaaacctggcacgcctggcaaataacccggaatacctggaacgtgcggaaaaaatttaccgtgaactg

SEQ ID NO: 9 - protein, 15 aa, synthetic construct
  source 1..15: note = hinge
EPKSGDKTHTCPPCP

SEQ ID NO: 10 - DNA, 45 bp, synthetic construct
  source 1..45: mol_type = other DNA; note = hinge
gagcccaaatctggtgacaaaactcacacatgcccaccgtgccca

SEQ ID NO: 11 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 651 bp, Homo sapiens
  source 1..651: mol_type = genomic DNA
gcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 13 - protein, 288 aa, synthetic construct
  source 1..288: note = PD-L1-3-hinge-CH2-CH3
DRERARELARILLKVIKLSDSPEARRQLLRNLEELAEKYKDPEVRRILEEAERYIKEPKSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - DNA, 864 bp, synthetic construct
  source 1..864: mol_type = other DNA; note = PD-L1-3-hinge-CH2-CH3
gaccgtgaacgtgcacgtgaactggctcgcattctgctgaaagtaattaaactgagcgactccccagaagcacgtcgtcagctgctgcgcaacctggaagaactggcggaaaaatataaagatccggaggttcgtcgtatcctggaagaagccgaacgttatatcaaagagcccaaatctggtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 15 - protein, 287 aa, synthetic construct
  source 1..287: note = PD-L1-1-hinge-CH2-CH3
SREAVRQLLEDARKSKDPELVRILLKVARNLAELLNDPELRRLVEEIEEILRRLREPKSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - DNA, 861 bp, synthetic construct
  source 1..861: mol_type = other DNA; note = PD-L1-1-hinge-CH2-CH3
agccgtgaagcagtacgtcagctgctggaagacgcacgtaaatctaaagacccggaactggtacgcatcctgctgaaggtggcacgtaacctggcggagctgctgaacgacccagaactgcgtcgtctggtggaagaaattgaagaaatcctgcgccgtctgcgtgagcccaaatctggtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 17 - protein, 291 aa, synthetic construct
  source 1..291: note = PD-L1-5-hinge-CH2-CH3
SAREEADRLLQEIARLRKEGDREKAEEIVKRLRELVERLNDPLLRIILKVAENILKELNEPKSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - DNA, 873 bp, synthetic construct
  source 1..873: mol_type = other DNA; note = PD-L1-5-hinge-CH2-CH3
tctgctcgtgaagaagctgatcgtctgctgcaggaaatcgctcgtctgcgcaaggaaggcgatcgtgaaaaagcagaagaaatcgtaaaacgtctgcgtgaactggttgaacgtctgaacgatccgctgctgcgtatcatcctgaaagttgctgaaaacatcctgaaggaactgaacgagcccaaatctggtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 19 - protein, 284 aa, synthetic construct
  source 1..284: note = PD-L1-2-hinge-CH2-CH3
SKEEALEQLLRDLKESTDPELIRILLKVIENLARLANNPEYLERAEKIYRELEPKSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - DNA, 852 bp, synthetic construct
  source 1..852: mol_type = other DNA; note = PD-L1-2-hinge-CH2-CH3
tctaaggaagaagctctggaacagctgctgcgcgatctgaaagaatctaccgatccggaactgatccgtattctgctgaaggttattgaaaacctggcacgcctggcaaataacccggaatacctggaacgtgcggaaaaaatttaccgtgaactggagcccaaatctggtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 21 - protein, 19 aa, synthetic construct
  source 1..19: note = Signal peptide
MGWSCIILFLVATATGVHS

SEQ ID NO: 22 - DNA, 57 bp, synthetic construct
  source 1..57: mol_type = other DNA; note = Signal peptide
atgggatggtcatgtatcatcctttttctagtagcaactgcaaccggtgtacattcc